(19) United States
(12) Patent Application Publication
(10) Pub. No.: US 2018/0000457 A1
(43) Pub. Date: Jan. 4, 2018

(54) DETERMINING POWER DIFFERENCE IN SENSOR SIGNALS

(71) Applicant: ENT. SERVICES DEVELOPMENT CORPORATION LP, Tyson, VA (US)

(72) Inventors: Larry SCHMIDT, Rutledge, GA (US); Satwant KAUR, Mountain View, CA (US)

(73) Assignee: ENT. SERVICES DEVELOPMENT CORPORATION LP, Tysons, VA (US)

(21) Appl. No.: 15/539,664

(22) PCT Filed: Dec. 23, 2014
(86) PCT No.: PCT/US2014/072022

§ 371 Date: Jun. 23, 2017

(51) Int. Cl. A61B 8/08; A61B 8/00

(52) U.S. Cl. CPC A61B 8/5215; A61B 8/4477; A61B 8/56

(57) ABSTRACT

Examples disclosed herein relate to determining a power difference in sensor signals. Examples include a first sensor to transmit a first ultrasonic signal into a pregnant woman and to receive a second ultrasonic signal; and a second sensor to transmit a third ultrasonic signal into the pregnant woman and to receive a fourth ultrasonic signal. A processing resource determines a first power difference of the first sensor according to a difference between respective powers of the first ultrasonic signal and the second ultrasonic signal and is to determine a second power difference of the second sensor according to a difference between respective power of the third ultrasonic signal and the fourth ultrasonic signal. In examples, the processing resource is to determine a relative location of the fetal heart according to a comparison of the first power difference and the second power difference.

### BACKGROUND

The health of a fetus may be monitored in the womb. Various techniques (listening for heartbeat, touching the womb) have been used for to monitor fetal progress. Modern medical equipment has allowed for monitoring of more fetal statistics.

### BRIEF DESCRIPTION OF THE DRAWINGS

The following detailed description references the drawings, wherein:

FIG. 1A is a partial schematic side perspective view of a monitor placed over a fetus according to an example.

FIG. 1B is a partial schematic side perspective view of a monitor placed over a fetus according to an example.

FIG. 2 is a schematic view of a monitor of FIG. 1A according to an example.

FIG. 3 is a schematic view of a monitor of FIG. 1B according to an example.

FIG. 4 is a flowchart of an example method for monitoring a fetus according to an example.

FIG. 5 is a flowchart of an example method for monitoring a fetus according to an example.

### DETAILED DESCRIPTION

In the following discussion and in the claims, the term “couple” or “couples” is intended to include suitable indirect and/or direct connections. Thus, if a first component is described as being coupled to a second component that coupling may, for example, be: (1) through a direct electrical or mechanical connection, (2) through an indirect electrical or mechanical connection via other devices and connections, (3) through an optical electrical connection, (4) through a wireless electrical connection, and/or (5) another suitable coupling.

Monitoring fetal vital signs is an important goal. Various devices have been developed to measure different vital signs of a fetus using various techniques. However, many of these devices are expensive to produce and need medical supervision to be used to monitor a fetus.

To address these issues, in the examples described herein, a fetal monitoring system is described that is cost effective and simple to operate with no supervision. The monitoring system includes a sensor array to emit ultrasonic signals into an expectant mother and received reflected ultrasonic signals. The monitoring system may determine the location of a fetus in the womb according to a difference in the respective powers of the transmitted ultrasonic signals and received ultrasonic signals. Other fetal vital signs may be determined by the monitoring system. In some examples, the monitoring system includes a transceiver to transmit the results of its analysis to a separate device to perform fetal monitoring. In such a manner, fetal vital signs can be monitored using a measurement of power only.

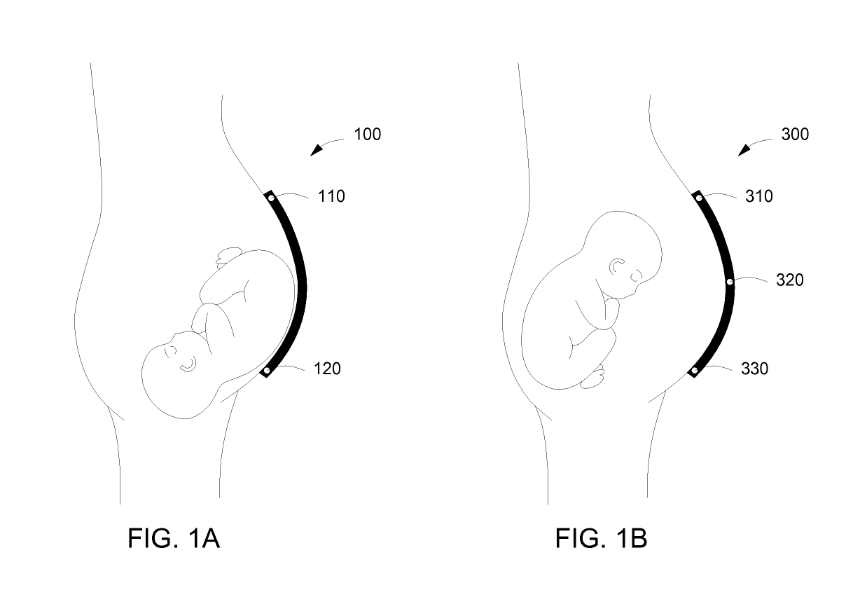

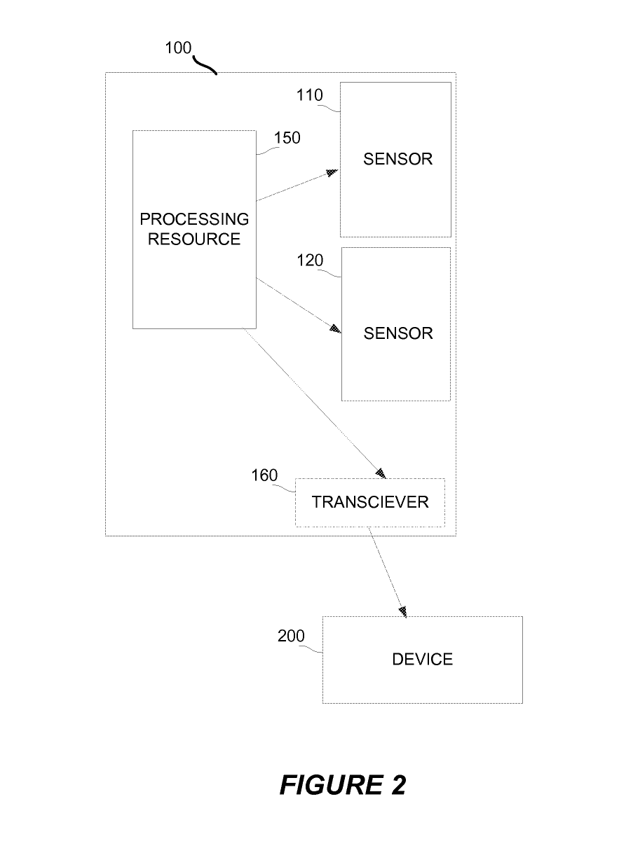

Referring now to the drawings, FIG. 1A is a partial schematic side perspective view of a monitor 100 placed over a fetus in the womb. FIG. 2 is a schematic view of monitor 100. Monitor 100 includes a sensor 110, a sensor 120, and a processing resource 150. In some examples, monitor 100 may also include a transceiver 160. Monitor 100 may be placed on an expectant mother to monitor a fetal heartbeat. In an example, the monitor 100 may be part of a flexible strap which may be positioned on a pregnant woman's belly. In some examples monitor 100 may communicate with a device 200 via transceiver 160. First device 200 may be any device to receive electronic signals, such as, a mobile phone, a smart watch, a table computer, a notebook computer, a desktop computer, a server, etc.

In an example, sensor 110 and sensor 120 may be an ultrasonic transducer/receiver to transmit ultrasonic signals and received ultrasonic signals. However, the examples are not limited thereto and sensor 110 and sensor 120 may be any type of sensor which transmits and receives signals. Although described as the same type of sensor, sensor 110 and sensor 120 may be different types of sensor from each other.

In examples described herein, the processing resource 150 may include, for example, one processor or multiple processors included in a single device or distributed across multiple computing devices. A “processor” may be at least one of a central processing unit (CPU), a semiconductor-based microprocessor, a graphics processing unit (GPU), a field-programmable gate array (FPGA) to retrieve and execute instructions, other electronic circuitry suitable for the retrieval and execution of instructions stored on a machine-readable storage medium, or a combination thereof. In other examples, the functionalities of any of the instructions stored on the machine-readable storage medium may be implemented in the form of electronic circuitry.

In examples, processing resource 150 may determine the location of a fetal heart and other vital statics of the fetus according to a power of signals transmitted and received by sensor 110. In an example, processing resource 150 may determine a difference in the power (hereafter “power difference) of a transmitted signal and a received signal at sensor 110 and sensor 120. The processing resource 150 may determine a location of a fetal heart relative to sensor 110 and sensor 120 according to the power difference. In an example, processing resource 150 may determine that a fetal heart is located closer to a sensor when there is a lower power difference between the transmitted signal and the received signal at that sensor compared to other sensors. The orientation of a fetus in the womb may be determined according to the fetal heat location. In some examples, if the fetus is in an orientation that may cause complications (e.g., a breech birth, etc.), processor 150 may transmit an alert via transceiver 160 to first device 200. First device 200 may provide an alarm to a user via a user interface device or may forward the alert to a medical professional. For example, first device 200 may transmit an SMS, an email, or other alarm message to a designated medical professional. However, the examples are not limited thereto and an alarm may be provided directly by monitor 100 to a pregnant woman. For example, monitor 100 may vibrate while on the mother's womb to indicate fetal distress. In such an example, the pregnant women may be alerted to seek out medical assistance for the fetal distress.

A fetal heartbeat constructively or destructively interferes with the ultrasonic signals transmitted by sensor 110 and sensor 120 and thus the reflected ultrasonic signals received by sensor 110 and sensor 120 will include interference pattern from the fetal heartbeat. The reflected ultrasonic signals generally lose signal strength as they travel back to sensor 110 or sensor 120 through the womb. Generally, the further an ultrasonic signal must travel, the more strength it will lose as it hits certain objects and portions of the signal are bounced off those objects before reaching sensor 110 or sensor 120. Thus, a sensor close to the fetal heart will receive ultrasonic signals that have traveled less distance than any other sensors. Processing resource 150 may determine a fetal heartbeat is present in the received ultrasonic signals by determining whether a frequency phase shift has occurred in received ultrasonic signals. Processing resource 150 may determine the location of the fetal heart by determining which sensor received an ultrasonic signal with the least amount of signal strength loss compared to the transmitted ultrasonic signal. In other words, the sensor with the least power difference between the transmitted signal and the received signals may be determined to be the closest to the fetal heart.

In some examples, monitor 100 may be continuously worn by an expectant mother to monitor fetal vital statics. In such an example, sensor 110 and sensor 120 may periodically transmit ultrasonic signals into the womb and receive ultrasonic signals from the womb. Processing resource 150 may monitor changes in fetal heat position, fetal heartbeat presence, etc. For example, the fetal monitor may monitor fetal location by periodically determine the location of fetal heart and alert the mother or medical professionals of the movement of the fetus, for example, into a birth position (e.g., when the fetal heart is located closer to sensor 110 in FIG. 1A), etc.

For example, with reference to FIG. 1A, processing resource 150 may determine the fetal heart is located closest to sensor 120 by determining the power difference at sensor 120 is less than the power difference at sensor 110. In such an example, ultrasonic signals transmitted by sensor 110 and sensor 120 travel into the womb and may constructively interfere with the sound of the fetal heartbeat. Some of the transmitted ultrasonic signals may be reflected from the fetus back to sensor 110 and sensor 120. Depending on the distance each signal travels, the strength of the ultrasonic signal at sensor 110 and sensor 120 will vary. In the example of FIG. 1A, the distance traveled by the ultrasonic signal received at sensor 120 after being reflected from and interfered with the fetal heartbeat is less than the distance an ultrasonic signal received by sensor 110 will travel. As a result, the ultrasonic signal received at sensor 120 will have less signal strength loss (i.e., power loss) compared to the transmitted ultrasound signal than the ultrasonic signals received at sensor 110.

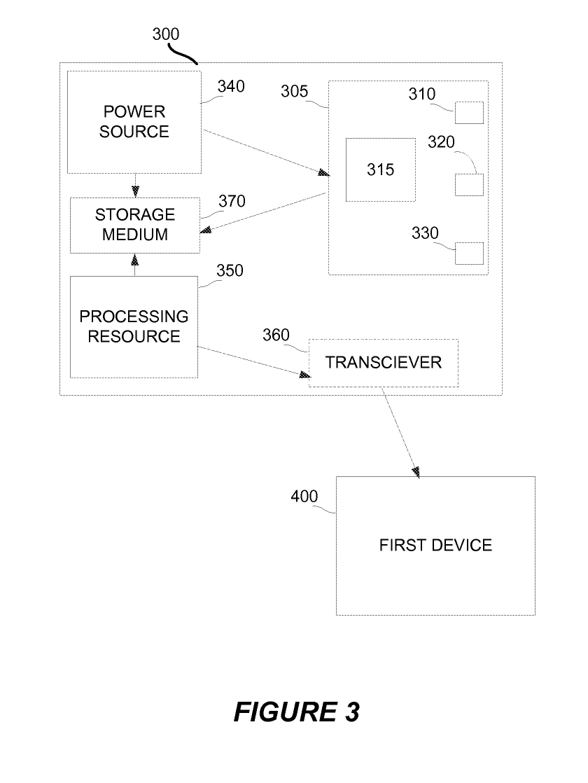

FIG. 1B is a partial schematic side perspective view of a monitor 300 placed over a fetus in the womb. FIG. 3 is a schematic view of monitor 300. Monitor 300 includes a sensor array 305, a power source 340, a processing resource 350, and a storage medium 370. In some examples, monitor 300 may include a transceiver 360. Monitor 300 may be placed on an expectant mother to monitor a fetal heartbeat. In an example, the monitor 300 may be part of a flexible strap which may be positioned on a pregnant woman's belly. In some examples monitor 300 may communicate with a first device 400 via transceiver 360. First device 400 may be any device to receive electronic signals, such as, a mobile phone, a smart watch, a table computer, a notebook computer, a desktop computer, a server, etc.

Sensor array 305 may include a power measurement unit 315, a sensor 310, a sensor 320, and a sensor 330. However the examples are not limited thereto and sensor array 305 may include any number of sensors, for example, a single sensor, two sensors, four sensors etc. In an example, sensor 310 may be an ultrasonic transducer/receiver to transmit ultrasonic signals and received ultrasonic signals. In some examples, sensor 320 and sensor 330 may also be ultrasonic transducers/receivers, however the examples are not limited thereto, and sensor 310, sensor 320, and sensor 330 may each be a different type of sensor.

Power measurement unit 315 may measure the power of a signal transmitted or received by any or all of the sensors of sensor array 305 (e.g., sensor 310, sensor 320, and sensor 330). In an example, ultrasonic signals received at sensor 310 are converted into mechanical energy and the mechanical energy is converted into electrical energy, and power measurement unit 315 may measure the electrical energy to determine the power of the ultrasonic signals. Similarly, the power measurement unit 315 may measure the electrical power provided by the power source 340 to the sensor array to transmit signals via sensor 310. An indicator of the electrical power measured by power measurement unit 315, whether from transmitted or received ultrasonic signals, may be stored in storage medium 370.

In an example, each of sensor 310, sensor 320, and sensor 330 may be ultrasonic transducer/receiver. In such an example, each of sensor 310, sensor 320, and sensor 330 may transmit ultrasonic signals into the womb of a pregnant women and receive reflected ultrasonic signals from the womb. However, the examples are not limited thereto, and sensor 310, sensor 320, and sensor 330 may be any sensor to transmit and receive any type of signal. In yet other examples, sensor 310, sensor 320, and sensor 330 may each be a different type of sensor.

Power source 340 may be any power source to provide power to monitor 300. Power source 300 may be a battery, a solar powered cell, a direct connection to another power source (i.e., wall jack), etc. In some examples, an indicator of the amount of power provided to the sensor array 305 to transmit signals may be stored in storage medium 370.

Processing resource 350 may fetch, decode, and execute instructions stored on storage medium 370 to perform the functionalities described below. In other examples, the functionalities of any of the instructions of storage medium 370 may be implemented in the form of electronic circuitry, in the form of executable instructions encoded on a machine-readable storage medium, or a combination thereof.

As used herein, a “machine-readable storage medium” may be any electronic, magnetic, optical, or other physical storage apparatus to contain or store information such as executable instructions, data, and the like. For example, any machine-readable storage medium described herein may be any of Random Access Memory (RAM), volatile memory, non-volatile memory, flash memory, a storage drive (e.g., a hard drive), a solid state drive, any type of storage disc (e.g., a compact disc, a DVD, etc.), and the like, or a combination thereof. Further, any machine-readable storage medium described herein may be non-transitory.

In examples, processing resource 350 may determine the location of a fetal heart and other vital statics of the fetus according to the measured power from power measurement unit 315 of sensor array 305. In an example, the processing resource 350 may determine the difference in the power of a transmitted signal and a received signal at each of sensor 310, sensor 320, and sensor 330. The processing resource 350 may determine a fetal heart is located closes to a sensor with the lowest power difference between the transmitted signal and the received signal. The orientation of a fetus in the womb may be determined according to the fetal heat location. In some examples, if the fetus is in an orientation that may cause complications (e.g., a breech birth, etc.), processor 350 may transmit an alert via transceiver 360 to first device 400. First device 400 may provide an alarm to a user via a user interface device or may forward the alert to a medical professional or midwife. For example, first device 400 may transmit an SMS, an email, or other alarm message to a designated medical professional. However, the examples are not limited thereto and an alarm may be provided directly by monitor 300 to a user. For example, monitor 300 may vibrate while on the mother's womb to indicate fetal distress. In such an example, the mother may be alerted to seek out medical assistance for the fetal distress.

A fetal heartbeat constructively or destructively interferes with the ultrasonic signals transmitted by the sensor array 305 and thus the reflected ultrasonic signals received by the sensor array 305 will include interference patter from the fetal heartbeat. A reflected ultrasonic signal generally loses signal strength as it travels back to a sensor through the womb. Generally, the further an ultrasonic signal must travel, the more strength it will lose as it hits certain objects and portions of the signal are bounced off those objects before reaching a sensor. Thus the sensor in sensor array 305 closest to the fetal heart will receive ultrasonic signals that have traveled less than any other sensor in sensor array 305. Processing resource 350 may determine a fetal heartbeat is present in the received ultrasonic signals by determining whether a frequency phase shift has occurred in received ultrasonic signals. Processing resource 350 may determine the location of the fetal heart by determining which sensor in sensor array 305 received an ultrasonic signal with the least amount of signal strength loss compared to the transmitted ultrasonic signal. In other words, the sensor in sensor array 305 which receives the highest powered ultrasonic signal may be determined to be the closest to the fetal heart.

For example, with reference to FIG. 1B, processing resource 350 may determine the fetal heart is located closest to sensor 310 by determining the power intensity difference at sensor 310 is less than the power intensity difference at sensor 320 and sensor 330. In such an example, ultrasonic signals transmitted by sensor 310, sensor 320, and sensor 330 travel into the womb and may destructively interfere with the sound of the fetal heartbeat. Some of the transmitted ultrasonic signals may be reflected from the fetus back to the sensor 310, sensor 320, and sensor 330. Depending on the distance each signal travels, the strength of the ultrasonic signal at each sensor will vary. In the example of FIG. 1B, the distance traveled by the ultrasonic signal received at sensor 310 after being reflected from and interfered with the fetal heart beat is less than the distance an ultrasonic signal received by sensor 320 and sensor 330 will travel. As a result, the ultrasonic signal received at sensor 310 will have less signal strength loss (i.e., power loss) compared to the transmitted ultrasound signal than the ultrasonic signals received at sensor 320 and sensor 330.

In some examples, monitor 300 may be continuously worn by an expectant mother to monitor fetal vital statics. In such an example, sensor array 305 may periodically transmit ultrasonic signals into the womb and receive ultrasonic signals from the womb. Processing resource 350 may monitor changes in fetal heat position, fetal heartbeat presence, etc. For example, the fetal monitor may monitor fetal location by periodically determining the location of fetal heart and alert the mother or medical professionals of the movement of the fetus, for example, into a birth position (e.g., when the fetal heart is located closest to sensor 310 in FIG. 1B), etc.

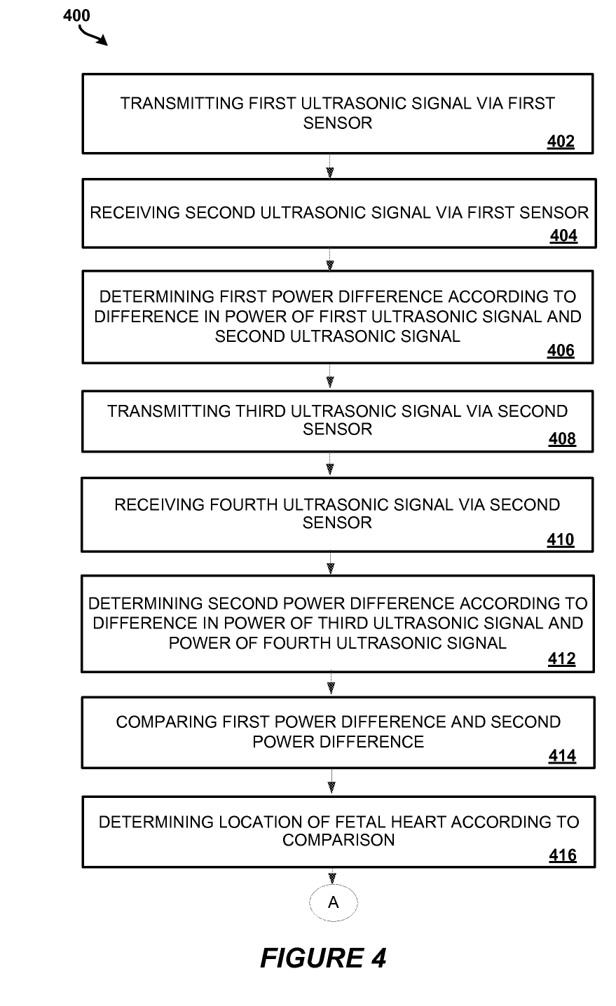

FIG. 4 is a flowchart of an example method 400 for monitoring a fetus according to an example. Although execution of method 400 is described below with reference to monitor 300 described above, other suitable systems (monitor 100) for the execution of method 400 can be utilized. Additionally, implementation of method 400 is not limited to such examples.

At 402 of method 400, monitor 300 transmits a first ultrasonic signal via sensor 310 into a pregnant woman.

At 404, monitor 300 receives a second ultrasonic signal via sensor 310 from the pregnant woman.

At 406, processing resource 350 determines a first power difference according to a difference in a power of the first ultrasonic signal and a power of the second ultrasonic signal.

At 408, monitor 300 transmits a third ultrasonic signal via sensor 320 into the pregnant woman.

At 410, monitor 300 receives a fourth ultrasonic signal via sensor 320 from the pregnant woman.

At 412, processing resource 350 determines a second power difference according to a difference in a power of the third ultrasonic signal and a power of the fourth ultrasonic signal.

At 414, processing resource 350 compares the first power difference with the second power difference.

At 416, processing resource 350 determines a location of a fetal heart according to the comparison at 414.

In some examples, method 400 includes transceiver 360 transmitting the location of the fetal heart to first device 400. In other examples, the method 400 includes transmitting an alert to the pregnant woman according to the determined location of the fetal heart, for example, via an alarm, vibration, etc.

Although the flowchart of FIG. 4 shows a specific order of performance of certain functionalities, method 400 is not limited to that order. For example, the functionalities shown in succession in the flowchart may be performed in a different order, may be executed concurrently or with partial concurrence, or a combination thereof. In some examples, functionalities described herein in relation to FIG. 4 may be provided in combination with functionalities described herein in relation to any of FIGS. 1-3.

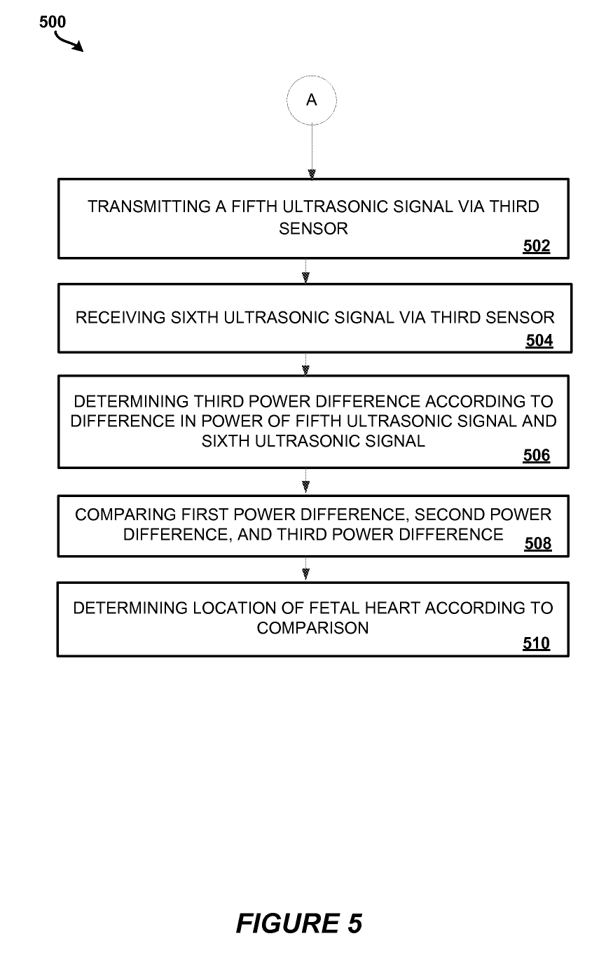

FIG. 5 is a flowchart of an example method 500 for monitoring a fetus according to an example. Although execution of method 500 is described below with reference to monitor 300 and method 400 described above, other suitable systems (monitor 100) for the execution of method 500 can be utilized. Additionally, implementation of method 500 is not limited to such examples.

In FIG. 5, method 500 includes 402 through 414 of method 400 and continues to 502.

At 502 of method 500, monitor 300 transmits a fifth ultrasonic signal via sensor 330 into a pregnant woman.

At 504, monitor 300 receives a sixth ultrasonic signal via sensor 330 from the pregnant woman.

At 506, processing resource 350 determines a third power difference according to a difference in a power of the fifth ultrasonic signal and a power of the sixth ultrasonic signal.

At 508, processing resource 350 compares the first power difference, the second power difference, and the third power difference.

At 510, processing resource 350 determines a location of a fetal heart according to the comparison at 508.

Although the flowchart of FIG. 5 shows a specific order of performance of certain functionalities, method 500 is not limited to that order. For example, the functionalities shown in succession in the flowchart may be performed in a different order, may be executed concurrently or with partial concurrence, or a combination thereof. In some examples, functionalities described herein in relation to FIG. 5 may be provided in combination with functionalities described herein in relation to any of FIGS. 1-4.

What is claimed is:

1. A system, comprising:
a first sensor to transmit a first ultrasonic signal into a pregnant woman and to receive a second ultrasonic signal; and
a second sensor to transmit a third ultrasonic signal into the pregnant woman and to receive a fourth ultrasonic signal, and
a processing resource to determine a first power difference of the first sensor according to a difference between respective powers of the first ultrasonic signal and the second ultrasonic signal, and to determine a second power difference of the second sensor according to a difference between respective powers of the third ultrasonic signal and the fourth ultrasonic signal;
wherein the processing resource is to determine a relative location of the fetal heart according to a comparison of the first power difference and the second power difference.

2. The system of claim 1, wherein the processing resource determines the relative location of the fetal heart is closer to the first sensor when the first power difference is less than the second power difference.

3. The system of claim 2, wherein the processing resource determines the relative location of the fetal heart is closer to the second sensor when the second power difference is less than the first power difference.

4. The system of claim 1, wherein the processor is to determine a frequency phase shift between the first ultrasonic signal and the second ultrasonic signal to determine if a fetal heart beat is present.

5. The system of claim 1, further comprising a transceiver to transmit the first power difference and the second power difference to a device.

6. An apparatus, comprising:
a ultrasound sensor array to transmit ultrasonic signals into a pregnant woman and receive ultrasonic signals;
a power source to provide power to the ultrasound sensor array; and
a processing resource to determine differences between power in the transmitted ultrasonic signals and power in the received ultrasonic signals,
wherein the processor is to determine a location of a fetal heart in relation to the ultrasound sensor array according to the differences in power.

7. The apparatus of claim 6, further comprising:
a transceiver to transmit the determined location of the fetal heart to a first device.

8. The apparatus of claim 6, wherein the ultrasound sensor array further comprises:
a power measurement unit to measure the power difference in the transmitted ultrasonic signals and the received ultrasonic signals.

9. The apparatus of claim 6, wherein the processor determines whether a fetal heart beat is present by analyzing a frequency phase shift of the received ultrasonic signals.

10. The apparatus of claim 6, further comprising a storage medium to store an indicator of the power provided to the ultrasound sensor array to transmit the ultrasonic signals.

11. The apparatus of claim 6, wherein the ultrasound sensor array is to periodically transmit the ultrasonic signals into the pregnant woman.

12. A method for detecting fetal distress, comprising:
transmitting a first ultrasonic signal via a first sensor into a pregnant woman;
receiving a second ultrasonic signal via the first sensor from the pregnant woman;
determining a first power difference according to a difference in a power of the first ultrasonic signal and a power of the second ultrasonic signal;
transmitting a third ultrasonic signal via a second sensor into the pregnant woman;
receiving a fourth ultrasonic signal via the second sensor from the pregnant woman;
determining a second power difference according to a difference in a power of the third ultrasonic signal and a power of the fourth ultrasonic signal;
comparing the first power difference and the second power difference; and
determining a location of a fetal heart according to the comparison.

13. The method claim 12, further comprising:
transmitting the location of the fetal heart to a first device.

14. The method claim 12, further comprising:
transmitting an alert to the pregnant woman according to the determined location of the fetal heart.

15. The method claim 12, further comprising:
transmitting a fifth ultrasonic signal via a third sensor into the pregnant woman;
receiving a sixth ultrasonic signal via the third sensor from the pregnant woman;
determining a third power difference according to a difference in a power of the fifth ultrasonic signal and a power of the sixth ultrasonic signal;
comparing the first power difference, the second power difference, and the third power difference; and
determining the location of the fetal heart according to the comparison.
